(19) United States
(12) Patent Application Publication
(10) Pub. No.: US 2020/0138140 A1
(43) Pub. Date: May 7, 2020

(54) Thermal Safety Helmet

(71) Applicant: Daniel M. Shapiro, Davie, FL (US)

(72) Inventor: Daniel M. Shapiro, Davie, FL (US)

(21) Appl. No.: 16/739,077

(22) Filed: Jan. 9, 2020

Related U.S. Application Data
(63) Continuation-in-part of application No. 15/712,028, filed on Sep. 21, 2017, now abandoned.
(60) Provisional application No. 62/397,806, filed on Sep. 21, 2016.

(51) Int. Cl. A42B 3/04; A42B 3/06; A61F 7/08

(52) U.S. Cl. CPC A42B 3/0406; A61F 2007/0002; A61F 7/08; A42B 3/06

(57) ABSTRACT

A pliable padded thermal safety helmet that conforms to a patient's head. The helmet has an exterior layer that protects a patient from impact injuries, and has an interior surface that secures removable thermal packs that provide hot or cold therapy depending on the treatment intended for an injury, infection, etc. The thermal packs are removably attached to the inside surface of the thermal safety helmet, via hook and loop material, at any desired location inside the thermal safety helmet to provide heat or cold to specific areas of a patient's head.

### CROSS-REFERENCE TO RELATED APPLICATIONS

This application is related to, and claims the benefit of, the now expired provisional patent application entitled “Thermal Safety Helmet For Treating And Prevention Of Head Injuries”, filed Sep. 21, 2016, bearing U.S. Ser. No. 62/397,806 and naming Daniel M. Shapiro, the named inventor herein, as sole inventor, and the co-pending non-provisional patent application entitled “Thermal Safety Helmet”, filed Sep. 9, 2017, bearing U.S. Ser. No. 15/712,028 and naming Daniel M. Shapiro, the named inventor herein, as sole inventor, the contents of both applications are specifically incorporated by reference herein in its entirety.

### BACKGROUND

### TECHNICAL FIELD

This invention relates in general to safety devices, and more particularly it relates to protective headgear that is worn to protect an individual's head while a head injury is healing, and to provide thermal control of the helmet to assist healing and recovery while protecting the patient's head from additional injury. Alternative embodiments of this invention use alternative materials to fabricate the exterior layer of the thermal safety helmet, including ethylene-vinyl acetate (“EVA”), open cell urethane foam, etc.

### BACKGROUND OF THE INVENTION

Padded helmets are commercially available to protect a wearer's head in a variety of situations. They are used by athletics in a variety of sports. These devices, such as football helmets, typically provide impact protection, but do nothing for temperature control. Likewise, padded helmets are available for treatment of a variety of medical conditions, most notably to protect patients with head injuries from inadvertently sustaining subsequent injuries. These medically oriented helmets also fail to provide thermal control mechanisms, especially methods of both cooling and/or warming depending on the needs of the individual patient.

Casual headwear, such as baseball caps, are known to have been used In conjunction with cooling devices for the purpose of keeping an individual cool and participating in outdoor activities. These devices typically use pre-cooled pouches containing ice, cold water, or gel that are positioned at the back of the wearer's head or around the head band. This type of headwear typically does not provide the wearer any protection, except from sunlight.

Some casual headwear even go so far as to provide water filled pouches that allow water to slowly be released on to a wearer's head such that heat from the wearer's body evaporates the water to create a cooling effect. Of these devices, some use a headband and a cap, and others use larger compartments that emit water over the entire wearer's head. These devices can provide relief from heat, but can also be uncomfortable due to the constant release of moisture onto a wearer's head.

Impact resistant headwear, such as sports helmets (e.g., football helmets), and protected medical headwear both provide impact protection. However, neither combine impact protection with thermal control elements that allow an individual's head to be either cooled or warmed at the individual's discretion. Further, the prior art does not provide a helmet that allows one or more thermal devices to be arbitrarily positioned anywhere on the inside of the helmet.

While the prior art has provided basic impact protection, it has failed to provide a protective helmet that simultaneously provides therapeutic properties, namely, the application of thermal treatments to any location on an individual's head.

### SUMMARY OF THE INVENTION

This invention provides a pliable padded helmet that conforms to a patient's head. The helmet is fabricated with an inner surface that is fabricated from material capable of removably securing thermal packs at any selected location on the inside of the helmet. The thermal packs can be hot or cold depending on the treatment intended for an injury, infection, etc. The thermal packs have an outer surface fabricated from soft pliant material that comes in contact with the patient's head. The helmet uses the thermal packs to selectively provide heat or cold to a selected location on a patient's head. The thermal packs have a soft material on the side that contacts the patient's head for comfort. Further, the helmet surface contacting patient's head is fabricated from material that is suitable as a hook and loop surface. Preferably, the material is fabricated from felt, felt-like, or other suitable material that is comfortable for the patient when worn. The side of the thermal pack that contacts the inside of the helmet has a hook and loop surface that allows the thermal pack to be securely and removably attached to any desired location on the inside of the helmet. Likewise, the side of the thermal pack that does not attach to the inside surface of the helmet is preferably fabricated from the same material as the inside surface of the helmet to maintain the same comfortable feel as the rest of the helmet.

An alternative embodiment of this invention provides a pliable EVA helmet that conforms to a patient's head. The helmet is fabricated as a single unit via a molding process. The EVA material provides a stable lightweight structure that allows it to be comfortably worn for extended periods of time. As was the case above, the inside surface is also hook and loop material that secures the thermal pack to the inside of the helmet as described above.

### BRIEF DESCRIPTION OF THE DRAWINGS

FIG. 1 is a front view of a preferred embodiment of the thermal safety helmet with optional ventilation apertures on the surface of the thermal safety helmet. A portion of the interior surface along with a portion of a thermal pack is also shown.

FIG. 2 is a top view of a preferred embodiment of the thermal safety helmet showing ventilation apertures on the upper surface of the helmet.

FIG. 3 is a left side view of a preferred embodiment of the thermal safety helmet showing an ear aperture, a strap attachment aperture, and optional ventilation apertures on the surface of the helmet.

FIG. 4 is a right side view of the preferred embodiment of the thermal safety helmet showing an ear aperture, a strap with optional chin pad, and optional ventilation apertures on the surface of the helmet.

FIG. 5 is a rear view of a preferred embodiment of the thermal safety helmet showing a strap attached to it, and optional ventilation apertures on the surface of the helmet.

FIG. 6 is an inside plan view of a preferred embodiment of the helmet showing how the thermal safety helmet can be stamped from a single sheet of material.

FIG. 7 is a cutaway side view of a preferred embodiment of the thermal safety helmet showing the interior surface of the thermal safety helmet covered with interior lining material.

FIG. 8 is a cutaway side view of a preferred embodiment of the thermal safety helmet illustrating the thermal safety packs, having different dimensions, secured to various selected locations of the interior surface of the helmet.

### DETAILED DESCRIPTION OF THE PREFERRED EMBODIMENT

Prior to a discussion of the figures, an overview of the invention will be presented. The invention is an apparatus and method for applying thermal treatments to head injuries or conditions while simultaneously providing impact protection for patients. The invention protects and treats injuries that have already occurred, and also protects the head from subsequent injuries. In some cases, as when children or adults have chronic or pre-existing conditions, the helmet may be worn prior to any actual injuries. The invention uses a pliable impact resistant shell that conforms to a patient's head such that the patient is protected. This is especially important for children who may not be as careful about protecting head injuries. When a child has a head injury, they may not be as careful as an adult, so that when they are playing or engaging in other activities, they may easily reinjure themselves. The helmet prevents reinjuring the wearer's head by providing impact protection. As a result, the wearer is protected from re-injury, which facilitates faster healing.

The helmet is fabricated with thermal packs that can be hot or cold depending on the treatment intended for an injury, an infection, etc. The invention uses thermal packs that can be positioned anywhere on the inside of the helmet. This is important since the location of an injury can vary from one individual to another. This is especially true for children. The preferred embodiment envisions removable internal thermal packs that allow them to be replaced as needed. The thermal packs provide heat or cold depending on the condition being treated.

The helmet can be fabricated from a variety of materials. The only requirement is that the materials chosen be suitable for the helmet's intended purpose. While a wide variety of materials can be used to fabricate the thermal safety helmet, a preferred material used in the preferred embodiment of the invention uses an open cell urethane foam. One example of an open cell urethane foam is commercially available Poron™ Plus foam cushioning. Open cell urethane foam provides several advantages. It can be stamped as a single piece during manufacture, and bonded together with an adhesive. Likewise, it can also be molded. Further, it also offers high rebound properties that are important in protecting patients from additional injuries when treating a previous injury or condition. It provides an open cell, reasonable structure that allows ventilation. In addition, it has antifungal properties that are important when treating head injuries that may be susceptible to infection. In the preferred embodiment, the helmet is envisioned as having an approximate thickness of 15 mm, but this can vary.

The interior of the helmet uses a surface of hook and loop material. In the preferred embodiment, the hook and loop material covers the entire interior surface of the helmet. This an important feature of the invention in that it allows the removable thermal packs to be attached to any location inside the helmet. This an improvement over prior art helmets using thermal devices in that they are limited to specific locations in the helmet, and as such, they are useless in treating injuries at other locations.

An alternative embodiment of the thermal safety helmet uses an EVA exterior layer fabricated as a single unit, preferably using a molding process.

For ease of discussion, unless otherwise noted herein, the materials open cell urethane foam, Poron and EVA shall be deemed to be interchangeable.

EVA has been discovered to exhibit highly desirable properties for use in helmets. Ethylene vinyl acetate (EVA) is the copolymer of ethylene and vinyl acetate. The ethylene typically comprises sixty to ninety percent of the EVA weight, while the vinyl acetate component usually comprises from ten to forty percent of the weight. EVA has desirable characteristics in that it is similar to elastomeric materials in softness and flexibility, but has an advantage in that it can be processed like numerous types of thermoplastics. EVA has a combination of qualities such as good clarity and gloss, low temperature toughness, adhesive properties, crack resistance, waterproof properties, and resistance to UV radiation. Its use in a helmet provides a valuable cushioning property that reduces the possibility of further impact injuries.

EVA is a component in a variety of products, including hot melt adhesives, hot glue sticks, plastic wraps, children's foam stickers, and medical applications (i.e., drug delivery devices). EVA is an expanded rubber (i.e., foam rubber). EVA is buoyant, and is useful in products that are intended to float. It can be used as an encapsulation material for silicon cells in the manufacture of photovoltaic modules. EVA, is also used in the formulation of paints.

The preferred embodiment envisions the use of a nylon chinstrap that is preferred due to its strength and durability properties. Likewise, the straps are commercially available and can be manufactured with integral hook and loop attachment surfaces. In addition, an optional cushioned sleeve can be provided such that, when the strap is secured to the patient, the chinstrap will be covered by the cushioned sleeve for the patient's comfort.

The shape of the helmet can also vary depending on the size of the patient as well as the type of affliction the helmet is intended to treat. Further, the optional chinstrap can be used to secure the helmet to the patient.

Having discussed the invention in general, we turn now to a detailed discussion of the drawings.

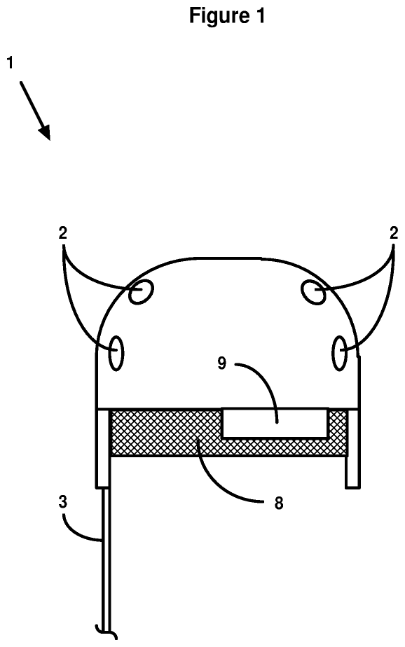

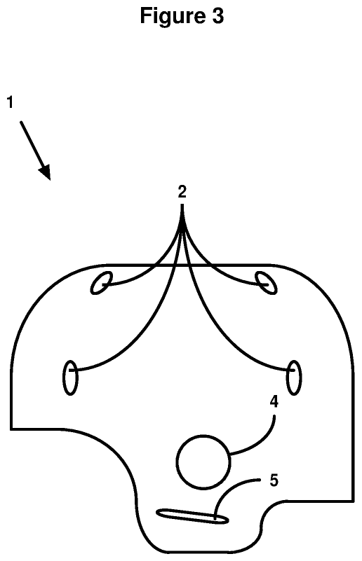

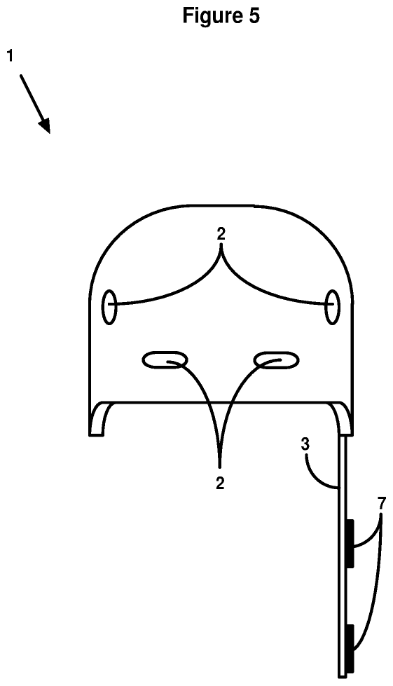

FIG. 1 is a front external view of a preferred embodiment of the thermal safety helmet 1 with optional ventilation apertures 2 on the surface of the thermal safety helmet 1. Also shown is a portion of the optional chinstrap 3 extending from the thermal safety helmet 1. In operation, the chinstrap 3 is inserted through chinstrap aperture 5 (shown in FIG. 3) and then preferably secured by integral hook and loop material 8 (shown in FIG. 5) on chinstrap 3. Any suitable method of securing the chinstrap 3 may be used, but hook and loop material 7 is preferred since it is inexpensive and easy to use. Those skilled in the art will recognize that the actual shape of the helmet 1 can vary based on the intended use and the patient's needs. Of course, the chinstrap 3 can be attached to either side of the thermal safety helmet 1.

Also shown in this figure is a portion of interior lining 8 which extends lower than the front of the thermal safety helmet 1. In the preferred embodiment, the interior lining 8 is fabricated from hook and loop material that may be felt, felt-like or any other material that is capable of securing a thermal pack 9 having a first side that is surfaced with hook and loop or a suitable alternative material. The material on the first side that faces the interior surface of the helmet 1 has hook and loop material to enable it to secure itself to the interior lining 8. The second side of the thermal pack 9 preferably has a surface that is fabricated from the same material that is used for interior lining 8. The use of the same material is to improve comfort for the wearer.

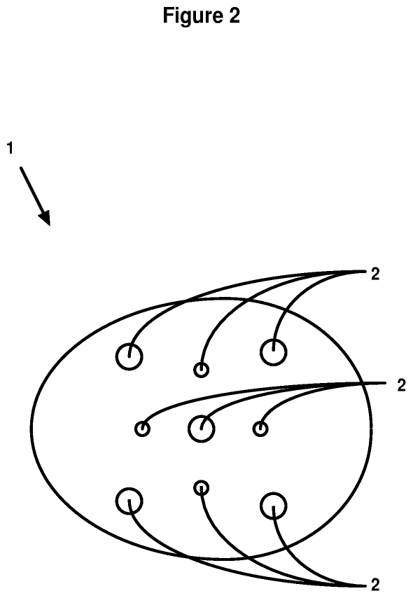

FIG. 2 is a top external view of a preferred embodiment of the thermal safety helmet 1 showing the optional ventilation apertures 2 on the upper surface of the thermal safety helmet 1. Those skilled in the art will realize that the number, size, and location of the ventilation apertures 2 can vary.

FIG. 3 is a left side view of a preferred embodiment of the thermal safety helmet 1 showing ear aperture 4, a strap attachment aperture 5, and the optional ventilation apertures 2 on the surface of the safety helmet 1. The ear aperture can vary in size and shape so long as it allows the wearer to hear sound from outside of the helmet 1.

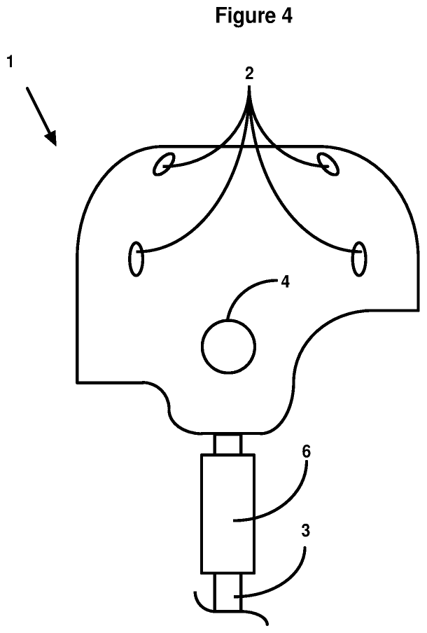

FIG. 4 is a right side view of the preferred embodiment of the thermal safety helmet 1 showing a second ear aperture 4, a chinstrap 3 with optional chin pad sleeve 6, and ventilation apertures 2 on the surface of the thermal safety helmet 1. Optional chinstrap sleeve 6 provides a soft and comfortable cushion for the patient's chin. Once the chinstrap 3 is inserted into strap aperture 5 and secured via hook and loop material 8, the chinstrap sleeve 6 is slidably moved over the chinstrap 3 such that the hook and loop material 8 (shown in FIG. 5) is covered up.

FIG. 5 is a rear view of a preferred embodiment of the thermal safety helmet 1 showing the chinstrap 3 attached to it, and optional ventilation apertures 2 on the surface of the thermal safety helmet 1. Also shown in this figure is the hook and loop material 7 on chinstrap 3.

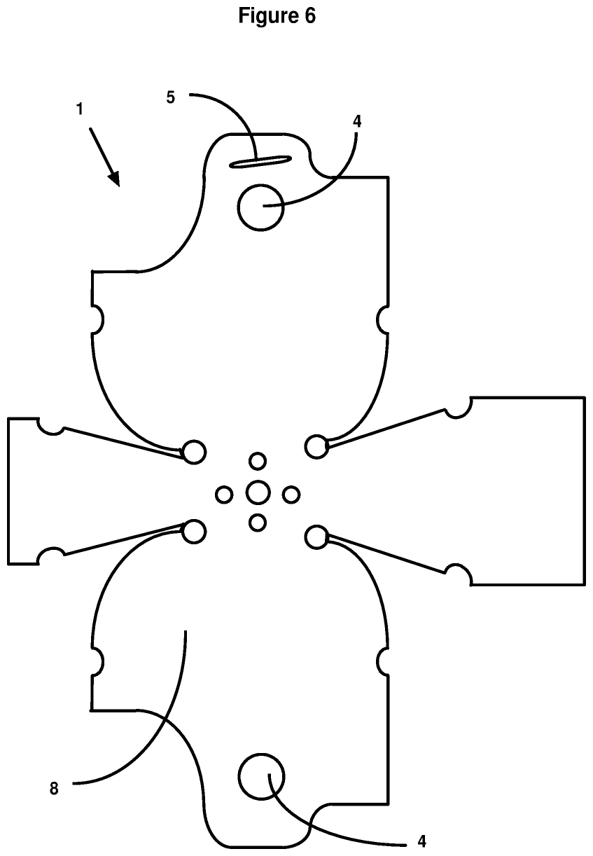

FIG. 6 is an inside plan view that illustrates how the thermal safety helmet 1 can be stamped from a single sheet of material. In a preferred embodiment, the interior lining 8 is designed to cover the entire interior of the thermal safety helmet 1. This allows a thermal pack 9 to be positioned anywhere on the interior of the thermal safety helmet 1. As a result, a medical professional or caretaker can position a thermal pack 9 at the precise location where the thermal pack 9 needs to be on the patient's head as the patient's needs dictate. Also illustrated in this figure are the chinstrap aperture 5, and the ear apertures 6. Those skilled in the art will recognize that the interior lining 8 can be attached to the sheets used to fabricate thermal safety helmet 1 prior to stamping, or secured to the thermal safety helmet 1 later, has in the case when using a molding process.

The thermal components of the thermal packs 9 can be fabricated from any suitable material. They can be commercially available “blue ice” packs, they can be chemical packs, etc. Further, the thermal packs can provide heat or cold thermal effects, as needed. In the preferred embodiment, the thermal pack is fabricated with a reusable non-toxic gel pack.

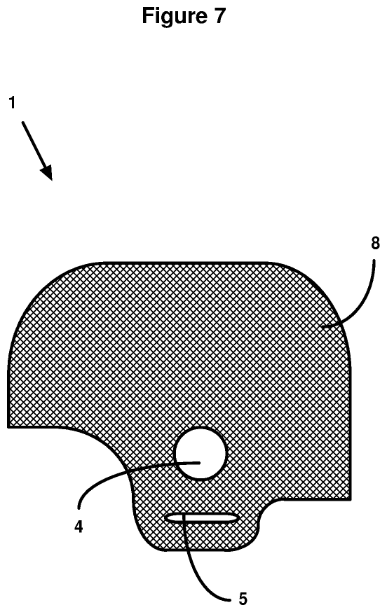

FIG. 7 is a cutaway side view of a preferred embodiment of the thermal safety helmet 1 showing the interior surface of the thermal safety helmet 1 covered with hook and loop material 8. Covering the entire inner surface of the thermal safety helmet 1 is preferred because it gives the health care provider the most flexibility in regard to placement of the thermal packs 9.

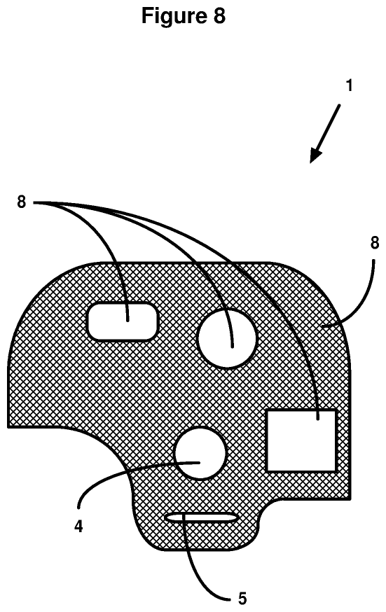

FIG. 8 is a cutaway side view of a preferred embodiment of the thermal safety helmet 1 illustrating how the thermal packs 9 in the thermal safety helmet 1 can be positioned at any desired location inside of the thermal safety helmet 1. In addition, this figure also illustrates the fact that the thermal packs 9 can be fabricated in any desired shape.

While specific embodiments have been discussed to illustrate the invention, it will be understood by those skilled in the art that variations in the embodiments can be made without departing from the spirit of the invention. For example, the materials used to fabricate the helmet can vary, the number and location of the thermal packs can vary, the method of securing the helmet to the patient's head can vary, etc. Therefore, the invention shall be limited solely to the scope of the claims.

I claim:

1. A thermal safety helmet for treating head injuries, comprising:
an exterior layer of impact resistant material that provides protection from external impacts;
an interior lining covering the entire interior surface of the helmet, the interior lining fabricated from material suitable for removable attachment to a thermal pack;
at least one detachable thermal pack, the thermal pack having a first side suitable for attachment to the interior lining;
the detachable thermal pack securable to any selected location on the interior lining such that it can be placed at the location of a head injury;
whereby, the thermal safety helmet provides impact protection to a patient and further provides thermal packs for the application of heat or cold to any selected area of a patient's head.

2. A thermal safety helmet, as in claim 1, wherein:
the interior lining is hook and loop material; and
the side of the thermal pack facing the interior lining is hook and loop material.

3. A thermal safety helmet, as in claim 1, wherein:
the exterior layer is fabricated from foam material.

4. A thermal safety helmet, as in claim 3, wherein:
the foam material is open cell urethane foam.

5. A thermal safety helmet, as in claim 1, wherein:
the exterior layer is fabricated from EVA material.
